(19) United States
(12) Patent Application Publication
(10) Pub. No.: US 2007/0016004 A1
(43) Pub. Date: Jan. 18, 2007

(54) Consumable unit and image forming device using the same

(75) Inventor: Joong-kil Kwon, Suwon-si (KR)

Correspondence Address: STEIN, MCEWEN & BUI, LLP, 1400 EYE STREET, NW, SUITE 300, WASHINGTON, DC, 20005, US

(73) Assignee: Samsung Electronics Co., Ltd., Suwon-si (KR)

(21) Appl. No.: 11/434,714

(22) Filed: May 17, 2006

(30) Foreign Application Priority Data
Jul. 4, 2005 (KR) 2005-59730

(51) Int. Cl. A61B 5/05

(52) U.S. Cl. 600/420

(57) ABSTRACT

A consumable unit attachable to and detachable from an image forming apparatus, including a consumable used to form an image, and a memory, including a non-reversible area, to store lifespan related information on the consumable at the non-reversible area thereof.

### CROSS-REFERENCE TO RELATED APPLICATIONS

This application claims the benefit of Korean Application No. 2005-59730, filed Jul. 4, 2005, in the Korean Intellectual Property Office, the disclosure of which is incorporated herein by reference.

### BACKGROUND OF THE INVENTION

1. Field of the Invention

Aspects of the present invention relates to a consumable unit and an image forming device using the same. More particularly, aspects of the present invention relate to an unrecyclable consumable unit and an image forming device using the same.

2. Description of the Related Art

Generally, an image forming apparatus comprehensively refers to Laser Beam Printers, LPHs (LED Print Heads), printers, facsimiles, and multifunctional apparatuses.

For printing, the image forming apparatus includes a plurality of articles of consumption units that need to be periodically or non-periodically replaced with new units. These include developing units, fixing units, and photosensitive drums, all of which have a limited lifespan.

The consumable units have a memory to maintain and manage the use of each, respectively. The memory stores with various types of information to maintain and manage the consumable units. Table 1 is a map showing information stored at the memory mounted at a conventional consumable unit.

TABLE 1

| Storage location | Capacity | | Stored Contents |
|---|---|---|---|
| 0x00 | 8 | Byte | Manufacturer's ID |
| 0x20 | 1 | Byte | Capacity of Toner Cartridge |
| 0x28 | 6 | Byte | Date of Manufacturer |
| 0x30 | 16 | Byte | Serial Number of Toner Cartridge |
| 0x40 | 8 | Byte | Date of Mounting |
| 0x48 | 4 | Byte | Printed Page Count |
| 0x58 | 8 | Byte | Toner (Dot) Count |
| 0x60 | 8 | Byte | Manufacturer's ID (Back-up) |
| 0x68 | 1 | Byte | Toner Level State |

When the consumable units, having memories to store the above information, are mounted at the image forming apparatus, a CPU (Central Processing Unit) of the image forming apparatus determines whether the corresponding consumable unit is genuine or not, based on the information stored at the mounted consumable unit. If the CPU determines that the consumable unit mounted at the image forming apparatus is not genuine, the CPU informs a user that a print quality can not be guaranteed.

The CPU of the image forming apparatus continues to observe the information stored in the memory of the consumables, to remind a user to replace the consumables with new consumables or controls the image forming apparatus not to operate, when the consumables are used up.

If a particular consumable is found to be used up, a user should replace the consumable with a new product. Because it may be hard for a general user to purchase a genuine product due to high prices, low-priced consumables that are recycled from unofficial lifespan-expired consumables are provided.

The memory mounted at the consumable is mainly an ordinary EEPROM (Electrically Erasable Programmable Read Only Memory) and it may not be hard to make the CPU of the image forming apparatus recognize the recyclable consumable as a genuine product or to reset the lifespan, by changing the information stored at the memory.

The CPU of the image forming apparatus, therefore, may have difficulty in determining whether the recyclable consumable, even one having the duplicated memory information of a genuine consumable, is genuine, or not. The use of non-genuine consumables or recyclable consumables may cause serious trouble in the image forming apparatus and further reduce the lifespan of the image forming apparatus.

### SUMMARY OF THE INVENTION

An aspect of the present invention is to solve at least the above and/or other problems and/or disadvantages of the related art and to provide at least the advantages described below. Accordingly, an aspect of the present invention is to provide an unrecyclable consumable unit and an image forming device using the same, by having a non-reversible area in a memory.

In order to achieve the above-described and/or other aspects of the present invention, there is provided a consumable unit, comprising: a consumable used to form an image; and a memory mounted at the consumable and storing information on the consumable at the non-reversible area.

The memory may further comprise the non-reversible area storing environment data out of information on the consumable. The non-reversible area may store progressive data out of the information on the consumable. The data on progress include lifespan data, printed page count, and dot count. The non-reversible area may store the information on the consumable in a predetermined non-reversible method of recording by bit unit. A Fuse Link method may be applied to the non-reversible method of recording, and a fuse corresponding to a predetermined bit at the non-reversible area may be severed to indicate a genuineness of the consumable. The consumable may one out of OPC (Organic Photo-Conductive) drum, a developing roller, a charging roller, a transferring roller, a fixing roller, a supplying roller, and a charging roller.

An image forming apparatus according to an embodiment of the present invention may comprises: at least one consumable unit having a consumable used for forming an image and a memory storing information on the consumable at a non-reversible area, and mounted attachable and detachable with respect to the image forming apparatus; and a controlling part controlling the consumable to drive by extracting the information on the consumable from the memory.

A non-reversible area may be stored with the information on the consumable according to a predetermined non-reversible method of recording based on bit. A Fuse Link method may be applied to the non-reversible method of recording, and the fuse corresponds to a predetermined bit at the non-reversible area that may be severed to indicate a genuineness of the consumable.

When the consumable unit is mounted, the controlling part may determine whether the consumable is genuine or not, based on the fuse connection at the non-reversible area. The controlling part may sequentially sever the fuse at the non-reversible area as the lifespan of the consumable progresses.

The image forming apparatus may further comprise a current supplying part supplying the current to the fuse. The controlling part may control the current supplying part to supply a current that is higher than an operating current to cut the fuse.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a block diagram of an image forming apparatus according to an embodiment of the present invention;

FIG. 2 shows a memory structure according to the embodiment of FIG. 1; and

FIG. 3 shows an internal structure of a non-reversible area of the memory of FIG. 2.

### DETAILED DESCRIPTION OF THE EMBODIMENTS

Reference will now be made in detail to the present embodiments of the present invention, examples of which are illustrated in the accompanying drawings, wherein like reference numerals refer to the like elements throughout. The embodiments are described below in order to explain the present invention by referring to the figures.

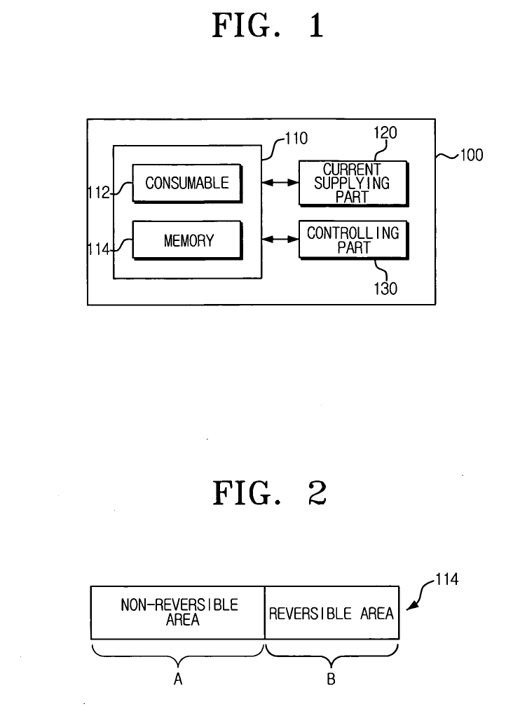

FIG. 1 is a block diagram of an image forming apparatus according to an embodiment of the present invention. As shown in FIG. 1, an image forming apparatus 100 performs printing through electrification, exposure, development, transfer, and fixation, and includes a consumable unit 110, a current supplying part 120, and a controlling part 130. The image forming apparatus 100 further comprise a displaying part, a main memory and a host interface, in addition to the consumable unit 110, the current supplying part 120, and the controlling part 130. They are already well-known and will not be described in detail.

The consumable unit 110 is mounted to be attachable to and detachable from the image forming apparatus 100 and needs to be replaced when the lifespan thereof expires. The consumable unit 110 requires the timely replacement to obtain clear prints and to prevent the image forming apparatus from being damaged. The consumable unit 110 includes a consumable 112 and a memory 114.

The consumable unit 110 is used to form an image. The consumable 110 may comprise an OPC (Organic Photo-Conductive) drum, a developing roller, a charging roller, a transferring roller, a fixing roller, a supplying roller and a pressurizing roller to perform electrification, exposure, development, transfer and fixation, all of which are needed to form the image.

The memory 114 is mounted at the location of the consumable 110 and stores information on the consumable. The information on the consumable may include an ID of the manufacturer, toner cartridge capacity, date of manufacture, printed page count, and toner count, as illustrated in Table 1.

The memory 114 may include a reversible area and a non-reversible area. The reversible area stores less important information, while the non-irreversible area stores highly important information. Here it is noted that the term “non-reversible” is to be understood as being substantially synonymous with terms such as “read-only,” “read-once,” “non-erasable,” and/or any other substantially similar words and phrases. The memory will be further described in detail with reference to FIG. 2.

The current supplying part 120 supplies a predetermined operating current according to the control of the controlling part 130. When the controlling part 130 checks the lifespan of the consumable unit 110, the current supplying part 120 may supply higher current than the general operating current under the control of the controlling part 130.

The controlling part 130 controls an overall function of the image forming apparatus 100 by a predetermined control program, and controls signal input and output between the consumable unit 110 and the current supplying part 120.

The controlling part 130 controls the consumable 112 to be driven by extracting information on the consumable that is stored in the memory 114. The controlling part 130 may determine whether the consumable unit 112 is genuine or not by a predetermined method.

When the consumable unit 110 is an initially mounted consumable unit of the image forming apparatus 100 when the image forming apparatus 100 is first assembled, the controlling part 130 only controls the consumable 112 to be driven. However, when the consumable unit 110 is an additionally supplied one of the image forming apparatus, the controlling part 130 determines whether the mounted consumable unit 110 is genuine or not.

When the controlling part 130 determines that the additionally supplied consumable unit is not genuine, the controlling part 130 may inform a user through the displaying part that the use of a non-genuine consumable unit 110 may cause the image forming apparatus 100 to operate improperly.

The controlling part 130 counts the printed pages every time printing is performed using the consumable unit 110, and stores the number of printed pages in the non-reversible area A (see FIG. 2) of the memory 114 so that the printed page count can not be reproduced or forged.

The controlling part 130 may inform the user of the need of replacing the consumable unit 110, when the number of printed pages is found to approach or reaches the lifespan of the consumable 112 as pre-registered in the memory.

FIG. 2 shows a memory structure. As illustrated in FIG. 2, the memory 114 includes the non-reversible area A and the reversible area B. However, various changes in form and details may be made therein from the spirit and scope of the invention. For example, the memory 114 may include the non-reversible area A only, or the storage area between the non-irreversible area A and the reversible area B may vary. That is, the ratio between the non-reversible area A and the reversible area B may be 50:50 or even 80:20.

The non-reversible area A stores what is determined to be highly important information of the consumable, so that the consumable unit itself is prevented from being rebuilt and so that a reproduction of the consumable unit 110 is prevented from being forged. For example, the non-reversible area stores progressive data such as lifespan data, printed page count data, and dot count data, which are highly likely to be used in a reproduction of the consumable unit 110. If information such as the lifespan data, the printed page count, and dot count are reset, an old consumable whose lifespan is expired may be reused. Accordingly, this information is stored at the non-reversible area A.

In contrast, the reversible area B stores environmental data, which are less influential and less important, even where data changes occur, than the types of data to be stored in the non-reversible area A described above. Further, the environmental data may need to be available to be called upon for reading and writing at any time. The reversible area B may use EEPROM mainly applied to a conventional memory 114. Generally, EEPROM enables a user to revise contents in the storage space, and uses a higher voltage than normal in order to repeatedly erase or re-record data.

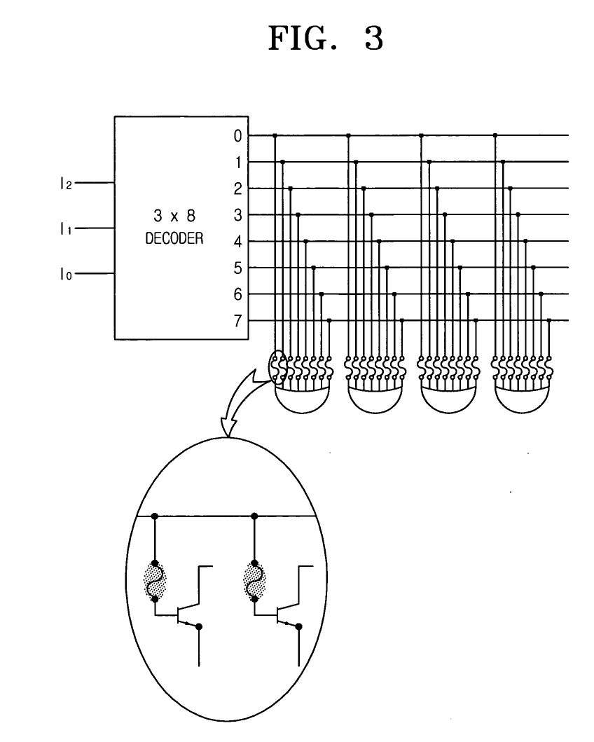

According to an embodiment of the present invention, information on the consumable is stored in the non-reversible area by bit unit in a predetermined non-reversible method of recording. The non-reversible method of recording may be a Fuse Link method. An embodiment of the Fuse Link method will be described in detail below with reference to FIG. 3, which illustrates an internal structure of a non-reversible area of the memory of FIG. 2.

As shown in FIG. 3, when the Fuse Link is used to record data at the non-reversible area A, each fuse is linked by bit unit according to a data record and, once-blown, a fuse can not be re-linked electrically and/or optically. Accordingly, it is impossible to change data stored at the non-reversible area A, at a disposal of a user.

When the Fuse Link is applied, the controlling part 130 blows the fuse by a bit when the lifespan of the consumable 112 is expired by 0.1%, so as to advance the count of the lifespan of the consumable 112.

Likewise, if data is recorded by applying the Fuse Link method, once blown, it is impossible to reset or restore information in the memory built at a genuine consumable. Conversely, resetting or restoration of memory is possible with conventional EEPROM memory methods.

As is mentioned above, a consumable unit and an image forming device using the same has a non-reversible area in a memory storage space in the consumable unit and stores important information on the lifespan of the consumable at the non-reversible area. As such, it is impossible to restore data stored in the non-reversible area in the memory or to rebuild/recycle a genuine consumable whose lifespan has expired. Furthermore, use of a reproduced consumable and/or an inferior consumable may be prevented by causing an image forming apparatus to recognize that a consumable unit inserted therein is possibly a reproduction of a consumable unit or a consumable unit that is being recycled and thereby issue a warning to a user.

Although a few embodiments of the present invention have been shown and described, it would be appreciated by those skilled in the art that changes may be made in this embodiment without departing from the principles and spirit of the invention, the scope of which is defined in the claims and their equivalents.

What is claimed is:

1. A consumable unit attachable to and detachable from an image forming apparatus, comprising;
a consumable used to form an image; and
a memory, including a non-reversible area, to store lifespan related information on the consumable at the non-reversible area thereof.

2. The consumable unit according to claim 1, wherein the memory comprises a reversible area to store environmental data on the consumable.

3. The consumable unit according to claim 1, wherein the lifespan related information comprises lifespan data, printed page count, and dot count.

4. The consumable unit of claim 1, wherein the lifespan related information is recorded in a non-reversible method by bit unit.

5. The consumable unit of claim 4, wherein the non-reversible method comprises a Fuse Link method.

6. The consumable unit of claim 4, wherein a fuse corresponding to a predetermined bit at the non-reversible area may be severed to indicate a genuineness of the consumable.

7. The consumable unit of claim 1, wherein the consumable comprise an OPC (Organic Photo-Conductive) drum, a developing roller, a charging roller, a transferring roller, a fixing roller, a supplying roller, and a pressurizing roller.

8. An image forming apparatus comprising:
at least one consumable unit having a consumable used to form an image and a memory, including a non-reversible area, to store information on the consumable at the non-reversible area, the consumable unit being attachable to and detachable from the image forming apparatus; and
a controlling part to control the consumable by extracting the information on the consumable from the memory.

9. The image forming apparatus of claim 8, wherein the lifespan related information is recorded in a non-reversible method by bit unit.

10. The image forming apparatus of claim 9, wherein the non-reversible method comprises a Fuse Link method.

11. The image forming apparatus of claim 10, wherein a fuse corresponding to a predetermined bit at the non-reversible are may be severed to indicated a genuineness of the consumable.

12. The image forming apparatus of claim 11, wherein when the consumable unit is mounted to the image forming apparatus, the controlling part determines whether the consumable is genuine based on the fuse connection at the non-reversible area.

13. The image forming apparatus of claim 10, wherein the controlling part sequentially severs the fuse at the non-reversible area as the lifespan of the consumable progresses.

14. The image forming apparatus of claim 13, further comprising a current supplying part to supply current to the fuse, wherein the controlling part controls the current supplying part to supply higher current than an operating current to sever the fuse.

15. The image forming apparatus of claim 8, wherein the consumable comprises an OPC (Organic Photo-Conductive) drum, a developing roller, a charging roller, a transferring roller, a fixing roller, a supplying roller, and a pressurizing roller.

16. An image forming apparatus to perform printing operations through electrification, exposure, development, transfer, and fixation, comprising:
a consumable unit, including a consumable and a memory, to be attachable to and detachable from the apparatus according to information stored in the memory relating to a lifespan of the consumable;
a current supplying part to supply an operating current to the consumable unit; and
a controlling part to regulate a supply of the operating current based on the information.

17. The image forming apparatus according to claim 16, wherein the information comprises an ID of the manufacturer of the consumable unit, a toner cartridge capacity, a date of manufacture, a printed page count, and a toner count.

18. The apparatus according to claim 16, wherein the memory comprises a reversible area to store relatively unimportant information relating to the consumable and a non-reversible area to store relatively important information relating to the consumable.

19. The apparatus according to claim 16, wherein the controlling part determines a genuineness of the consumable unit based on the information.

20. An image forming apparatus to perform printing operations through electrification, exposure, development, transfer and fixation, comprising:
a consumable unit, including a consumable and a memory, to be attachable to and detachable from the apparatus according to information store in the memory relating to a lifespan of the consumable;
a current supplying part to supply an operating current to the consumable unit; and
a controlling part to temporarily increase a supply of the operating current each time the printing operations are performed so as to cause the information relating to the lifespan of the consumable to progress.
